(19) United States
(12) Patent Application Publication
(10) Pub. No.: US 2009/0281398 A1
(43) Pub. Date: Nov. 12, 2009

(54) SYSTEMS, COMPOSITIONS AND METHODS FOR PSYCHOMETRIC ASSESSMENT OF COGNITIVE RECOVERY AFTER ANESTHESIA

(76) Inventor: Kirk J. Hogan, Madison, WI (US)

Correspondence Address: Casimir Jones, S.C., 440 Science Drive, Suite 203, Madison, WI, 53711, US

(21) Appl. No.: 12/463,267

(22) Filed: May 8, 2009

Related U.S. Application Data
(60) Provisional application No. 61/051,598, filed on May 8, 2008.

(51) Int. Cl. A61B 5/00; A63F 9/24

(52) U.S. Cl. 600/301; 463/7

(57) ABSTRACT

The present invention relates to recovery from anesthesia. In particular, the present invention relates to systems, compositions and methods of monitoring and assessing recovery of cognitive function in subjects undergoing surgical and medical procedures requiring anesthesia and sedation.

### CROSS-REFERENCE TO RELATED APPLICATIONS

This application claims priority to United State Provisional Application Ser. No. 61/051,598, filed May 8, 2008, the disclosure of which is herein incorporated by reference in its entirety.

### FIELD OF THE INVENTION

The present invention relates to recovery from anesthesia. In particular, the present invention relates to systems, compositions and methods of monitoring and assessing recovery of cognitive function in subjects undergoing surgical and medical procedures requiring anesthesia and sedation.

### BACKGROUND

The modified Aldrete scoring system is a commonly used tool for determining patient readiness for discharge from a post-anesthesia, or post-sedation, recovery facility. The Aldrete score was first developed in 1970 when most drugs used to provide anesthesia, amnesia and sedation were long-acting, and slow to reverse even after delivery was discontinued. (Aldrete JA, Modifications to the postanesthesia score for use in ambulatory surgery. J Perianesth Nurs. 1998 June; 13(3):148-55.). At that time, patients were uniformly admitted to the hospital for observation after surgery. The modified Aldrete Score incorporates multiple indices of general patient welfare including stability of vital signs, oxygenation, adequate control of pain and nausea, hydration, and the return of motor skills. The modified Aldrete score is noteworthy, however, for its deficient sensitivity to recovery of cognitive function including, for example, orientation, near- and long-term memory, attention, concentration, calculation, executive function and learning. Post anesthesia recovery scoring systems devised more recently share these defects.

Premature discharge of a patient from a post-anesthesia or post-sedation care unit in view of persistent cognitive impairments after medical or surgical procedures puts patients at risk for harm, and may result in caregiver and institutional liability. Conversely, delayed discharge after the patient is mentally “street-ready” (i.e., waiting until the patient's duration of stay is in compliance with one-size-fits-all, time-based discharge criteria) decreases patient satisfaction, and is wasteful of costly facility and personnel resources. Because nursing and personnel costs account for the majority of post-anesthesia care unit expenditures, with only 2% of costs allocated to medication and supplies, a robust and reproducible means for identify cognitive recovery is both time and cost saving. To avoid premature, or inappropriately delayed discharge, caregivers and institutions must ensure that the patient is “street ready” prior to discharge, that there is appropriate documentation of cognitive recovery, that the patient has learned and is able to recall instructions for self-care and assisted care outside of the facility, and that the specified discharge criteria have been met. Accordingly, there is great need for methods for monitoring and assessing recovery of cognitive function in subjects undergoing surgical and medical procedures requiring anesthesia and sedation.

### SUMMARY

The present invention relates to recovery from anesthesia. In particular, the present invention relates to systems, compositions and methods of monitoring and assessing recovery of cognitive function in subjects undergoing surgical and medical procedures requiring anesthesia and sedation.

In certain embodiments, the present invention provides an apparatus for assessing cognitive recovery from anesthesia in a subject, comprising a computing device, comprising a processor; a memory; a means for inputting data; a means for displaying data; and a computing program installed on the memory configured to assess cognitive recovery from anesthesia wherein the program accepts data in response to queries regarding at least two of the subject's orientation, near- and long-term memory, attention, concentration, calculation, executive function and learning. In some embodiments, the program accepts data in response to queries regarding at least two of said subject's orientation to time, orientation to place, visuo-spatial orientation, immediate recall and delayed recall. In other embodiments, the computing device is a hand-held computing device. In further embodiments, cognitive recovery is 70%, 80%, 90% or 100% cognitive recovery. In still further embodiments, the queries comprise baseline queries before anesthesia and serial queries after anesthesia. In certain embodiments, recovery comprises a score based on said responses to said queries. In preferred embodiments, the score comprises a threshold score for discharge from a post-anesthesia care facility. In particular embodiments, the anesthesia is general anesthesia. In other embodiments, the anesthesia is regional anesthesia. In further embodiments, the anesthesia is combined general and regional anesthesia. In some embodiments, the anesthesia is monitored anesthesia care. In still other embodiments, the anesthesia is deep sedation, moderate sedation, or minimal sedation. In further embodiments, the recovery is recovery from intensive care unit sedation. In some embodiments, the means for inputting data is a keyboard, touchscreen or mouse. In other embodiments, the means for inputting data is a microphone. In still other embodiments, the means for inputting data is a caregiver, family member, assistant or attendant. In further embodiments, the means for inputting data is an automated electronic data sharing system

In some embodiments of the apparatus of the present invention, the queries are selected from the group consisting of queries of verbal comprehension, perceptual organization, executive function, visual tracking, game performance, psychomotor performance, psychomotor speed, learning, memory, digital symbol substitution, attention, concentration, processing speed, dot-connection, flicker-fusion, simple reaction time, choice reaction time, p-deletion, and perceptive accuracy. In other embodiments, the queries of executive function further comprise queries of abstraction, problem solving and cognitive flexibility. In further embodiments, the computer program corrects for short-term learning. In still further embodiments, the computer program learns from the subject's responses. In certain embodiments, the queries further comprise queries regarding anesthesia data and surgical data. In some embodiments, the anesthesia data comprises data relating to drugs, doses and complications. In preferred embodiments, the means for displaying data is a CRT or video screen. In other embodiments, the means for displaying data is an auditory response. In still other embodiments, the means for displaying data comprises a print copy of the data. In further embodiments, the means for displaying data comprises an electronic copy of the data. In some embodiments, the program is installed on computer readable media. In certain embodiments, the queries are configured in accordance with the subject's age, gender, health, personal preference and procedure. In some embodiments the queries further comprise queries of the subject's comprehension of post-operative instructions.

In some embodiments, the present invention provides an electronically scored anesthesia recovery game, comprising: an electronic controller; a memory comprising two or more game narratives comprising two or more tests configured to assess cognitive recovery from anesthesia comprising verbal comprehension, perceptual organization, executive function, visual tracking, game performance, psychomotor performance, psychomotor speed, learning, memory, digital symbol substitution, attention, concentration, processing speed, dot-connection, flicker-fusion, simple reaction time, choice reaction time, p-deletion, perceptive accuracy, abstraction, problem solving and cognitive flexibility; a game narrative switch; at least one sensor operatively connected with the controller, the sensor adapted to detect at least two or more activities associated with the two or more tests and to generate two or more signals; and a game memory adapted to generate a user's cumulative test score from the two or more signals. In some embodiments, the game is selected from a group consisting of an electronic audio game, an electronic video game, a combined electronic audio and video game, and an interactive digital game. In other embodiments, the cumulative test score is calculated from signals obtained before anesthesia and surgery, and at pre-determined intervals after anesthesia and surgery. In some embodiments, the game narrative may be selected by said player. In other embodiments, the game further comprises instructions for playing the game. In still other embodiments, visual feedback is provided on a visual device and/or audio feedback is provided on an audio device. In some embodiments, the present invention comprises a personal computer, a personal digital assistant (PDA), IPOD, IPHONE, a game console, or game platform. In other embodiments, the present invention comprises an input device comprising at least one button, at least one joystick, a keyboard or a mouse. In further embodiments, the present invention comprises a haptic peripheral device providing vibration, force, or somatosensory feedback.

In some embodiments, the present invention provides a method for assessing cognitive recovery from anesthesia, comprising: providing an anesthesia care subject; providing an apparatus for assessing cognitive recovery from anesthesia, comprising: a computing device, comprising: a processor; a memory; a means for inputting data; a means for displaying data; and a computing program installed on the memory configured to assess cognitive recovery from anesthesia, wherein the program accepts data in response to queries regarding at least two of the subject's orientation, near- and long-term memory, attention, concentration, calculation, executive function and learning; and determining cognitive recovery from anesthesia from responses to queries from the subject. In other embodiments, the determining step further comprises comparing the subject's responses to the queries before and after anesthesia. In still other embodiments, the determining step further comprises calculation of a response score. In further embodiments, the determining step further comprises serial calculation of response scores. In particular embodiments, the display means comprises a web-enabled, electronic medical record. In some embodiments, the anesthesia care subject is a patient. In other embodiments, the anesthesia care subject is a caregiver. In further embodiments, the determining step further comprises comparing the subject's responses to the queries to responses in a database. In still further embodiments, the apparatus further comprises an electronic audio game, an electronic video game, a combined electronic audio and video game, or an interactive digital game. In preferred embodiments, the anesthesia care is selected from the group consisting of general anesthesia, regional anesthesia, monitored anesthesia care, awake deep sedation, moderate sedation, minimal sedation, acute sedation, chronic sedation and combinations thereof.

In other embodiments, the computing program further comprises instructions for use. In some embodiments, the computing program is self-operated. In further embodiments, the subject's responses to the queries are compared between responses before anesthesia and at two or more predetermined intervals after anesthesia. In particularly preferred embodiments, the predetermined interval comprises 5 minutes, 10 minutes, 15 minutes, 30 minutes or 60 minutes.

### DEFINITIONS

To facilitate an understanding of the present invention, a number of terms and phrases are defined below:

As used herein the terms “processor,” “digital signal processor,” “DSP,” “central processing unit” or “CPU” are used interchangeably and refer to a device that is able to read a program (e.g., algorithm) and perform a set of steps according to the program.

As used herein, the term “algorithm” refers to a procedure devised to perform a function.

As used herein, the term “audio signal” refers to any kind of audible noise, including, but not limited to musical pieces, speeches, and natural sounds.

As used herein, the term “per time interval” refers to any increment of time (e.g., milliseconds, seconds, minutes, hours, days, months).

As used herein, the term “audio player” refers to any kind of device or system capable of presenting (e.g., playing) an audio signal. Examples of audio players include, but are not limited to, I-Pods, mini-disc players, mp3 players, walkmans, in-ear audio devices, and digital audio players.

As used herein, the term “video player” refers to any kind of device or system capable of presenting (e.g., playing) a video signal. Examples of video players include, but are not limited to, television and computer monitors, hand-held video devices, cellular phones, I-Pods, video-display goggles, and the like.

### DETAILED DESCRIPTION

The present invention relates to recovery from anesthesia. In particular, the present invention relates to systems, compositions and methods of monitoring and assessing recovery of cognitive function in subjects undergoing surgical and medical procedures requiring anesthesia and sedation.

Recently a number of newer anesthetic drugs (e.g., desflurane, sevoflurane, remifentanil, propofol, xenon), and techniques (e.g., minimally invasive surgery, use of laryngeal mask airways) have been introduced into practice that facilitate rapid recovery from anesthesia, result in little or no “hangover”, and may engender minimal impact on acute or prolonged mental dysfunction, although this has not been fully established. No suitable tests are presently administered, or even contemplated, for routine use after medical and surgical procedures to assess the neuropsychological or psychometric characteristics of a patient needed to properly manage patient release after a procedure. Perhaps the most significant factor delaying translation of effective psychometric evaluation to the perioperative setting is the present day lack of hardware, software and methods configured specifically for use in the post-anesthesia care setting.

Accordingly, the hardware, software and methods of the present application are designed to fill this need. In one embodiment of the present invention, patients undergoing a medical or surgical procedure requiring the use of drugs that alter mental status are provided a series of self-administered tests of cognitive function on, for example, a hand-held personal digital assistant (PDA), IPOD, IPHONE, or similar device to be performed as a baseline measure before drug administration. Performance on multiple dimensions of cognitive function are tested including, for example, the patient's orientation to time and place, immediate recall, delayed recall, attention, executive function, reaction time, concentration and learning. Specific tests are selected from, for example, tests of verbal comprehension, perceptual organization, executive function (abstraction, problem solving and cognitive flexibility), visual tracking, game performance, psychomotor performance, psychomotor speed, digital symbol substitution, processing speed, dot-connection, flicker-fusion, simple reaction time, choice reaction time and perceptive accuracy. In some embodiments, performance test are configured for the detection of postoperative delirium. In other embodiments, performance tests are configured for the detection of post-operative cognitive dysfunction (POCD). As used herein, postoperative cognitive dysfunction refers to refers to new onset or worsening of preoperative cognitive problems with, for example, memory, learning and the ability to concentrate) following surgery. The onset of POCD may first appear days, weeks or months after surgery, and the effects may be long-lasting or permanent. Accordingly, in some embodiments performance tests of the present invention are configured to detect abnormalities upon serial testing days, weeks or months after surgery. In other embodiments, psychometric performance tests of the present invention are used to evaluate interventions for the prevention or therapy of POCD. In further embodiments, psychometric performance tests for the detection of POCD are games-based psychometric performance tests.

In other embodiments, psychometric performance tests of the present invention are configured to detect the onset or progression of Alzheimer's disease after surgery or anesthesia. In additional embodiments, psychometric performance tests of the present invention are configured to detect the onset or progression of cognitive dysfunction after cancer chemotherapy. In further embodiments, psychometric tests configured for detection of the onset or progression of Alzheimer's disease after surgery or anesthesia, or for the detection of cognitive dysfunction after cancer chemotherapy, are games-based psychometric performance tests.

After the procedure, and after the patient has satisfied intermediate non-cognitive recovery criteria for discharge using the Modified Aldrete Score, the patient again serially performs self-administered tests of cognitive function on the PDA, IPOD, IPHONE, or similar device. Patients meeting threshold performance scores in relation to personal baseline scores are discharged from observation. A subject with scores falling below threshold (i.e., between cognitive scores recorded at baseline and after surgery) are held for further observation. In circumstances wherein new and persistent cognitive deficits become apparent on serial testing, a subject is admitted to a facility and referred for additional neurologic and neuropsychologic testing to establish the timing of the lesion, its etiology, differential diagnosis and prognosis.

In some embodiments, the hardware, software and methods of the present invention are used in routine care. In other embodiments, the present invention is used as an index to compare recovery times between agents and techniques in clinical research, for example, to screen for compounds and/or techniques that provide improved benefits for patients or sub-sets of patients (e.g., patients of a particular age, gender, weight, genetic profile, etc.). In other embodiments, measures of cognitive performance after surgery and anesthesia are compared with other indices of recovery, for example, central nervous system imaging and electrophysiology. In preferred embodiments, the patient inputs data by a keyboard, touchscreen, mouse or microphone, roller ball, microphone, writing utensil, joystick, video game controller, steering wheel, musical instrument (e.g., drum, keyboard, guitar, etc.), floor pad (e.g., with one or more foot location sensors), balance platform, sports device (e.g., tennis racket, golf club) or other controller or peripheral device. In other embodiments, the systems, compositions and methods of the present invention are configured for specific patients, specific surgical, anesthetic and medical procedures, specific drugs and combinations of drugs, specific anesthetic techniques and regimens, and for specific populations of patients. In other embodiments, instructions, queries and cues are provided to the patient by display screen, speaker, touch or by earphone. In further embodiments, data acquired from the patient is algorithmically compared to the patient's pre-operative score, and a calculated score is compared to thresholds established for discharge from the post-anesthesia care facility, or is compared to a database wherein such test results are archived, collated, and thresholds for discharge are updated and improved. In some embodiments, a hard copy or electronic copy of the patient's serial psychometric test performance is placed in the patient's hard or electronic medical record, together with details of the anesthesia and procedure. In other embodiments, the hardware, software and methods of the present application are used to provide the patient with instructions for post-operative care, and to test and document the patient's comprehension and recall of these instructions. In certain embodiments, the hardware, software and methods of the present application are used to test and document caregiver readiness to deliver anesthesia and perform surgery.

In preferred embodiments, the systems of the present invention comprise a user interface. In preferred embodiments, the user interface is a graphical user interface. In particularly preferred embodiments, the graphical user interface provides numerous control elements for a user. For example, in preferred embodiments, the graphical user interface provides a slider control for simulating the rate of movement of a user. In yet other preferred embodiments, the graphical user interface provides an “about” box for instructions in the operation and use of the present invention. Additionally, the graphical user interface is capable of launching web browsers for accessing information or data from a particular web site. In further preferred embodiments, the graphical user interface provides an options dialog for the purpose of fine tuning the algorithm. The present invention is not limited to a particular type of user interface. In some embodiments, the design of the user interface is in the form of a Microsoft Foundation Classes (hereinafter, “MFC”)-based Windows graphical user interface.

In some embodiments, the testing system, device, or method challenges the patient with one or more games or activity simulations to monitor or assess one or more of orientation to time and place, immediate recall, delayed recall, attention, executive function, reaction time, concentration and learning. Specific tests are selected from, for example, tests of verbal comprehension, perceptual organization, executive function (abstraction, problem solving and cognitive flexibility), visual tracking, game performance, psychomotor performance, psychomotor speed, digital symbol substitution, processing speed, dot-connection, flicker-fusion, simple reaction time, choice reaction time and perceptive accuracy. In other embodiments, the testing system, device or method of the present invention tests memory of previously-learned materials by assessing recall of facts, terms, basic concepts and answers. In some embodiments, tests of knowledge may include tests of terminology, specific facts, ways and means of dealing with specific conventions, trends, sequences, classifications, categories, criteria and methods. In certain embodiments, tests of knowledge may include tests of abstraction and universal principles, generalizations, theories and structures. In further embodiments, the testing system, device or method of the present invention tests comprehension of facts and ideas in tests of organizing, comparing, translating, interpreting, providing descriptions or restating ideas, including tests of translation, interpretation and extrapolation. In still further embodiments, the testing system, device or method of the present invention tests applying new knowledge to solve problems by, for example, applying acquired knowledge, facts, techniques, rules and the like in a fashion different from that in which the new knowledge was acquired. In other embodiments, the testing system, device or method tests a user's analytical ability to examine and fragment information into component parts by, for example, identification of motivations or causes, making inferences, identification of evidence to support generalizations, and analysis of elements, relationships and organizational principles. In some embodiments, the testing system, device or method tests the user's ability to synthesize information in a different fashion by combining elements in new patterns, or deriving new solutions to problems including, for example, producing a new communication, plan, prediction, set of operations, or set of abstract relationships. In other embodiments, the testing system, device or method of the present invention tests the user's ability to make judgments about information, the validity of ideas, or evaluate internal evidence and external criteria. In additional embodiments the testing system, device or method of the present invention tests the user's affective skills in attitudes, emotion and feelings in, for example, paying attention, learning, attaching value to an object, phenomenon, or information, comparing, relating and elaborating on learned material, and identification of a particular value or belief. In preferred embodiments, the testing system, device or method tests the user's ability to physically manipulate a tool or instrument.

### Experimental Example

The following Example is provided in order to demonstrate and further illustrate certain preferred embodiments and aspects of the present invention and is not to be construed as limiting the scope thereof.

In one embodiment of the present invention, a subject is scheduled to undergo general anesthesia for an elective orthopedic procedure. During the subject's scheduled pre-surgical evaluation the subject is provided with a menu of electronically scored anesthesia recovery game narratives encoded in software installed on a personal digital assistant. Using a game narrative switch, the subject chooses to be assessed with a car driving/racing simulation digital interactive game that challenges the subject with increasing degrees of difficulty on multiple tasks such as braking, cornering, obstacle course driving, avoiding other vehicles, and driving in simulated adverse conditions (e.g., darkness, rain, snow, icy surface, loose surface, etc.). In some embodiments, existing software programs that collect performance data are used, such as, for example, GRAND TURISMO. In some embodiments, the patient is further tested with language games, pattern recognition games, sporting games, physical coordination games, and the like relating to the subject matter of the subject's chosen narrative.

After establishing a baseline pre-anesthesia, awake cumulative test score, the subject undergoes general anesthesia and surgery. When the surgery is completed, anesthesia is discontinued, the patient is allowed to wake up in the operating room, and to recover in the post-anesthesia care unit (PACU). In the PACU the patient is assessed for, for example, movement, respiratory effort, blood pressure, sedation level and oxygen saturation according to the modified Aldrete score. When the patient has achieved a cumulative modified Aldrete score that is stable over 15 minute intervals and has returned to pre-anesthesia baseline levels, the subject is provided with the car driving/racing simulation digital interactive game on the PDA, IPOD, or IPHONE. Over serial performance of the self-administered car driving game at 15 minute intervals the subject's electronically scored anesthesia recovery game cumulative test score returns to baseline and the subject is discharged from the anesthesia and surgery care facility. The electronically scored anesthesia recovery game provides the subject with post-anesthesia care instructions, and tests the subject for comprehension and recall of the instructions. Copies of the subject's baseline and post-anesthesia cumulative electronically scored anesthesia recovery game performance scores, and comprehension and memory of post-anesthesia care instructions, are placed on the subject's hard-copy and electronic medical records.

All publications and patents mentioned in the above specification are herein incorporated by reference. The foregoing description of several expressions of embodiments and methods of the invention has been presented for the purposes of illustration. The description is not intended to be exhaustive or to limit the invention to the precise forms and procedures described, and many modifications and variations are possible in light of the above description. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields, are intended to be within the scope of the following claims.

I claim:

1. An apparatus for assessing cognitive recovery from anesthesia in a subject, comprising:
a computing device, comprising:
a processor;
a memory;
a means for inputting data;
a means for displaying data; and
a computing program installed on said memory configured to assess cognitive recovery from anesthesia wherein said program accepts data in response to queries regarding at least two of said subject's orientation to time, orientation to place, visuo-spatial orientation, near- and long-term memory, immediate recall and delayed recall attention, concentration, calculation, executive function and learning.

2. The apparatus of claim 1, wherein said computing device is a hand-held computing device.

3. The apparatus of claim 1, wherein said cognitive recovery is 70%, 80%, 90% or 100% cognitive recovery.

4. The method of claim 1, wherein said anesthesia is selected from the group consisting of general anesthesia, regional anesthesia, monitored anesthesia care, awake deep sedation, moderate sedation, minimal sedation, acute sedation, chronic sedation, intensive care unit sedation and combinations thereof.

5. The apparatus of claim 1, wherein said means for inputting data is a keyboard, touchscreen or mouse.

6. The apparatus of claim 1, wherein said queries are selected from the group consisting of queries of verbal comprehension, perceptual organization, executive function, visual tracking, game performance, psychomotor performance, psychomotor speed, learning, memory, digital symbol substitution, attention, concentration, processing speed, dot-connection, flicker-fusion, simple reaction time, choice reaction time, p-deletion, queries of abstraction, problem solving and cognitive flexibility and perceptive accuracy.

7. The apparatus of claim 1, wherein said computer program corrects for short-term learning.

8. The apparatus of claim 1, wherein said means for displaying data comprises a CRT, a video screen, a print copy of said data or an electronic copy of said data.

9. The apparatus of claim 1, wherein said program is installed on a computer readable medium.

10. The apparatus of claim 1, wherein said queries further comprise queries of said subject's comprehension of post-operative instructions.

11. An electronically scored anesthesia recovery game, comprising:
an electronic controller;
a memory comprising two or more game narratives comprising two or more tests configured to assess cognitive recovery from anesthesia comprising verbal comprehension, perceptual organization, executive function, visual tracking, game performance, psychomotor performance, psychomotor speed, learning, memory, digital symbol substitution, attention, concentration, processing speed, dot-connection, flicker-fusion, simple reaction time, choice reaction time, p-deletion, perceptive accuracy, abstraction, problem solving and cognitive flexibility;
a game narrative switch;
at least one sensor operatively connected with said controller, said sensor adapted to detect at least two or more activities associated with said two or more tests and to generate two or more signals; and
a game memory adapted to generate a user's cumulative test score from said two or more signals.

12. The electronically scored anesthesia recovery game of claim 11, wherein said game is selected from a group consisting of an electronic audio game, an electronic video game, a combined electronic audio and video game, and an interactive digital game.

13. The electronically scored anesthesia recovery game of claim 11, wherein said cumulative test score is calculated from signals obtained before anesthesia and surgery and at pre-determined intervals after said anesthesia and surgery.

14. The electronically scored anesthesia recovery game of claim 11, wherein said game narrative may be selected by said player.

15. The electronically scored anesthesia recovery game of claim 11, further comprising visual feedback on a visual device and/or audio feedback on an audio device.

16. A method for assessing cognitive recovery from anesthesia, comprising:
providing an anesthesia care subject;
providing an apparatus for assessing cognitive recovery from anesthesia, comprising:
a computing device, comprising:
a processor;
a memory;
a means for inputting data;
a means for displaying data; and
a computing program installed on said memory configured to assess cognitive recovery from anesthesia, wherein said program accepts data in response to queries regarding at least two of said subject's orientation, near- and long-term memory, attention, concentration, calculation, executive function and learning; and
determining said cognitive recovery from anesthesia from said data in response to said queries from said subject.

17. The method of claim 16, wherein said subject's responses to said queries are compared between responses before anesthesia and at two or more predetermined intervals after anesthesia.

18. The method of claim 16, wherein said anesthesia care subject comprises a patient or a caregiver.

19. The method of claim 16, wherein said apparatus further comprises an electronic audio game, an electronic video game, a combined electronic audio and video game, and an interactive digital game.

20. The method of claim 16, wherein said computing program further comprises instructions for use.
